(19) United States
(12) Patent Application Publication
(10) Pub. No.: US 2023/0167109 A1
(43) Pub. Date: Jun. 1, 2023

(54) Aminopiperidine Amides, Derivatives, Compositions, and Uses Related to CXCR4 Modulation

(71) Applicant: Emory University, Atlanta, GA (US)

(72) Inventors: Hyunsuk Shim, Atlanta, GA (US); Yoon Hyeun Oum, Marietta, GA (US)

(21) Appl. No.: 17/921,051

(22) PCT Filed: Apr. 26, 2021
(86) PCT No.: PCT/US2021/029192

§ 371 Date: Oct. 24, 2022

Related U.S. Application Data
(60) Provisional application No. 63/015,082, filed on Apr. 24, 2020.

(51) Int. Cl. C07D 471/04; A61P 29/00

(52) U.S. Cl. CPC C07D 471/04; A61P 29/00

(57) ABSTRACT

This disclosure relates to aminopiperidine amides, derivatives, pharmaceutical compositions, and uses related to CXCR4 modulation. In certain embodiments, the aminopiperidine amides are compounds having formula I, salts, derivatives, and prodrugs thereof wherein, R¹, R², and R³ are further defined herein. In certain embodiments, this disclosure contemplates pharmaceutical compositions comprising compounds disclosed herein. In certain embodiments, this disclosure relates to methods of treating or preventing CXCR4 related diseases or conditions by administering an effective amount of a compound disclosed herein to a subject in need thereof.

### CROSS-REFERENCE TO RELATED APPLICATIONS

This application claims the benefit of U.S. Provisional Application No. 63/015,082 filed Apr. 24, 2020. The entirety of this application is hereby incorporated by reference for all purposes.

### BACKGROUND

C-X-C chemokine receptor type 4 (CXCR4), also known as fusin or cluster of differentiation 184 (CD184), is a seven transmembrane G-protein coupled receptor (GPCR). C-X-C chemokine ligand 12 (CXCL12) is the major ligand of CXCR4 and the interaction between CXCL12 and CXCR4 directs cells to tissues with high levels of CXCL12 expression. CXCR4 is overexpressed in many human cancer types. Accumulating evidence suggests the involvement of CXCR4-CXCL12 interactions in various inflammatory diseases, including rheumatoid arthritis, autoimmune diseases, ischemic injuries, inflammatory bowel disease, and pneumonia. Based on these findings, development of CXCR4 modulators presents a therapeutic strategy in inflammatory diseases as well as cancer.

Shu et al. report the inhibition of the CXCL12/CXCR4-axis as preventive therapy for radiation-induced pulmonary fibrosis. PLoS ONE, 2013, 8(11): e79768.

Liang et al. report small molecule modulators of CXCR4. PLoS ONE, 2012, 7(4): e34038. Bai et al. report symmetrical bis-tertiary amines as CXCR4 inhibitors. Eur J Med Chem 2016, 118, 340-350. Mishra et al. report characterization of CXCR4 receptor agonists and antagonists. Sci. Rep., 2016, 6, 30155.

Cumming et al. report N-4-piperidinyl compounds as CCR5 modulators. See WO2004018425.

References cited herein are not an admission of prior art.

### SUMMARY

This disclosure relates to aminopiperidine amides, derivatives, pharmaceutical compositions, and uses related to CXCR4 modulation. In certain embodiments, the aminopiperidine amides are compounds having formula I,

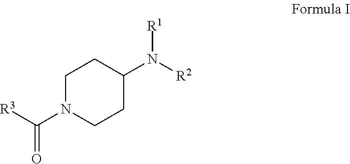

salts, derivatives, and prodrugs thereof wherein, R¹, R², and R³ are further defined herein. In certain embodiments, this disclosure contemplates pharmaceutical compositions comprising compounds disclosed herein. In certain embodiments, this disclosure relates to methods of treating or preventing CXCR4 related diseases or conditions by administering an effective amount of a compound disclosed herein to a subject in need thereof.

In certain embodiments, the disclosure relates to any compounds disclosed herein or derivatives optionally substituted with one or more substituents.

In certain embodiments, the disclosure relates to pharmaceutical composition comprising a compound disclosed herein or salt or derivative or prodrug thereof and a pharmaceutically acceptable excipient, diluent, or carrier. In certain embodiments, the pharmaceutical composition further comprises another active ingredient.

In certain embodiments, the disclosure relates to methods of treating or preventing an inflammatory condition, radiation-induced pulmonary fibrosis, cancer, microbial infection, or enhancing the mobilization of hematopoietic stem/progenitor cells from the bone marrow to the peripheral blood comprising administering a compound disclosed herein or pharmaceutical composition comprising the same to a subject in need thereof.

In certain embodiments, the disclosure relates to methods of treating or preventing an inflammatory condition comprising administering pharmaceutical composition comprising a compound disclosed herein in combination with another active ingredient to a subject in need thereof.

In certain embodiments, the disclosure relates to methods treating or preventing radiation-induced pulmonary fibrosis comprising administering pharmaceutical composition comprising a compound as disclosed herein in combination with another active ingredient to a subject in need thereof.

In certain embodiments, the disclosure relates to uses of a compound disclosed herein in the production of a medicament for the treatment of a CXCR4 related condition.

In certain embodiments, the disclosure relates to methods of making compounds disclosed herein comprising mixing a starting material disclosed herein with a reactant and/or reagents under conditions such that the products are formed.

### BRIEF DESCRIPTION OF THE SEVERAL VIEWS OF THE DRAWINGS

FIG. 1A shows a synthetic scheme for preparing compounds disclosed herein such as Z7R; (a) NaH, DMF, 1 h RT; (b) 24 h RT, 55%; (c) LiOH, MeOH:H2O=1:1, 12 h RT; (d) HOBt, EDCI, TEA, DMF, 12 h RT, 85%; (e) TFA:TIPS=95:5, DCM; (f) HOBt, EDCI, TEA, DMF, 12 RT, 70%; (g) TFA:TIPS:H2O=90:5:5.

FIG. 1B illustrates a scheme for preparing Z7R derivatives.

FIG. 2A shows data for compounds of this disclosure. EC (effective concentration) was calculated as the concentration at which the compound blocks >50% of CXCR4-targeted peptidomimetic conjugate (rhodamine Red X-TN14003). The inhibition % against the chemoattract, CXCL12 was determined by Matrigel invasion assay using 100 nM of compounds.

FIG. 2B shows data for compounds Z7R and plerixafor (AMD3100).

FIG. 3 shows computer calculations for the interactions of compounds of this disclosure and CXCR4. Z7X: X stands for side chain of amino acid X. MMGBSA ΔGbind is the calculated binding free energy from XP docking pose using molecular mechanics-generalized Born surface area method.

FIG. 4 shows data on Z7R-treated mice indicating the reduction of paw weight (— 50%) in the carrageenan-induced paw edema lesion, compared with the untreated mice. Acute paw inflammation was induced by subcutaneous injection of 50 μL of λ-carrageenan in left hind paw. The mice in the treatment group were all administered Z7R at 10 mg/kg i.p., while control animals received corresponding i.p. injection of vehicle.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

Certain of the compounds described herein may contain one or more asymmetric centers and may give rise to enantiomers, diastereomers, and other stereoisomeric forms that can be defined, in terms of absolute stereochemistry at each asymmetric atom, as (R)— or (S)—. The present chemical entities, pharmaceutical compositions and methods are meant to include all such possible isomers, including racemic mixtures, tautomer forms, hydrated forms, optically substantially pure forms and intermediate mixtures.

Unless otherwise stated, structures depicted herein are also meant to include compounds which differ only in the presence of one or more isotopically enriched atoms. For example, compounds having the present structures except for the replacement or enrichment of a hydrogen by deuterium or tritium at one or more atoms in the molecule, or the replacement or enrichment of a carbon by ¹³C or ¹⁴C at one or more atoms in the molecule, are within the scope of this disclosure. In one embodiment, provided herein are isotopically labeled compounds having one or more hydrogen atoms replaced by or enriched by deuterium. In one embodiment, provided herein are isotopically labeled compounds having one or more hydrogen atoms replaced by or enriched by tritium. In one embodiment, provided herein are isotopically labeled compounds having one or more carbon atoms replaced or enriched by ¹³C. In one embodiment, provided herein are isotopically labeled compounds having one or more carbon atoms replaced or enriched by ¹⁴C.

The disclosure also embraces isotopically labeled compounds which are identical to those recited herein, except that one or more atoms are replaced by an atom having an atomic mass or mass number different from the atomic mass or mass number usually found in nature. Examples of isotopes that can be incorporated into disclosed compounds include isotopes of hydrogen, carbon, nitrogen, oxygen, phosphorus, sulfur, fluorine, and chlorine, such as, e.g., ²H, ³H, ¹³C, ¹⁴C, ¹⁵N, ¹⁸O, ¹⁷O, ³¹P, ³²P, ³⁵S, ¹⁸F, and ³⁶Cl, respectively. Certain isotopically-labeled disclosed compounds (e.g., those labeled with ³H and/or ¹⁴C) are useful in compound and/or substrate tissue distribution assays. Tritiated (i.e., ³H) and carbon-14 (i.e., ¹⁴C) isotopes can allow for ease of preparation and detectability. Further, substitution with heavier isotopes such as deuterium (i.e., ²H) can afford certain therapeutic advantages resulting from greater metabolic stability (e.g., increased in vivo half-life or reduced dosage requirements). Isotopically labeled disclosed compounds can generally be prepared by substituting an isotopically labeled reagent for a non-isotopically labeled reagent. In some embodiments, provided herein are compounds that can also contain unnatural proportions of atomic isotopes at one or more of atoms that constitute such compounds. All isotopic variations of the compounds as disclosed herein, whether radioactive or not, are encompassed within the scope of the present disclosure.

As used herein, “alkyl” means a noncyclic straight chain or branched, unsaturated or saturated hydrocarbon such as those containing from 1 to 22 carbon atoms, while the term “lower alkyl” or “C₁₋₄alkyl” has the same meaning as alkyl but contains from 1 to 4 carbon atoms. The term “higher alkyl” has the same meaning as alkyl but contains from 8 to 22 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or heterocarbocyclyl” groups are carbocycles which contain from 1 to 4 heteroatoms independently selected from nitrogen, oxygen and sulfur which may be saturated or unsaturated (but not aromatic), monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Aryl” means an aromatic carbocyclic monocyclic or polycyclic ring such as phenyl or naphthyl. Polycyclic ring systems may, but are not required to, contain one or more non-aromatic rings, as long as one of the rings is aromatic. “Arylalkyl” means an alkyl substituted with an aryl, e.g., benzyl, methyl substituted with phenyl.

As used herein, “heteroaryl” refers to an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

“Alkylthio” refers to an alkyl group as defined above attached through a sulfur bridge. An example of an alkylthio is methylthio, (i.e., —S—CH₃).

“Alkoxy” refers to an alkyl group as defined above attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, propoxy, n-butoxy, s-butoxy, and t-butoxy.

“Alkylamino” refers to an alkyl group as defined above attached through an amino bridge. An example of an alkylamino is methylamino, (i.e., —NH—CH₃).

“Alkanoyl” refers to an alkyl as defined above attached through a carbonyl bridge (i.e., —(C═O)alkyl).

“Alkylsulfonyl” refers to an alkyl as defined above attached through a sulfonyl bridge (i.e., —S(═O)₂alkyl) such as mesyl and the like, and “Arylsulfonyl” refers to an aryl attached through a sulfonyl bridge (i.e., —S(═O)₂aryl).

“Alkylsulfinyl” refers to an alkyl as defined above attached through a sulfinyl bridge (i.e. —S(═O)alkyl).

“Aminoalkyl” refers to an amino group attached through an alkyl bridge. An example of an aminoalkyl is aminomethyl, (i.e., NH₂—CH₂—).

“Hydroxyalkyl” refers to a hydroxy group attached through an alkyl bridge. An example of a hydroxyalkyl is hydroxyethyl, (i.e., HO—CH₂CH₂—).

“Guanidinoalkyl” refers to a guanidino group attached through an alkyl bridge. An example of a guanidinoalkyl is guanidinopropyl, (i.e., NH₂C(═N)NH—CH₂CH₂ CH₂—).

“Heterocyclylalkyl” refers to a heterocycle attached through an alkyl bridge. An example of a heterocyclylalkyl is imidazolylmethyl, (i.e., imidazolyl-CH₂—).

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

The term “optionally substituted,” as used herein, means that substitution is optional and therefore it is possible for the designated atom to be unsubstituted.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In typical embodiments, the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

“Subject” refers to any animal, preferably a human patient, livestock, rodent, monkey or domestic pet.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis.

Typical prodrugs are pharmaceutically acceptable esters. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

As used herein, “pharmaceutically acceptable esters” include, but are not limited to, alkyl, alkenyl, alkynyl, aryl, arylalkyl, and cycloalkyl esters of acidic groups, including, but not limited to, carboxylic acids, phosphoric acids, phosphinic acids, sulfonic acids, sulfinic acids, and boronic acids.

As used herein, the term “derivative” refers to a structurally similar compound that retains sufficient functional attributes of the identified analogue. The derivative may be structurally similar because it is lacking one or more atoms, substituted with one or more substituents, a salt, in different hydration/oxidation states, e.g., substituting a single or double bond, substituting a hydroxy group for a ketone, or because one or more atoms within the molecule are switched, such as, but not limited to, replacing an oxygen atom with a sulfur or nitrogen atom or replacing an amino group with a hydroxyl group or vice versa. Replacing a carbon with nitrogen in an aromatic ring is a contemplated derivative. The derivative may be a prodrug. Derivatives may be prepared by any variety of synthetic methods or appropriate adaptations presented in the chemical literature or as in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

The cancer to be treated in the context of the present disclosure may be any type of cancer or tumor. These tumors or cancer include, and are not limited to, tumors of the hematopoietic and lymphoid tissues or hematopoietic and lymphoid malignancies, tumors that affect the blood, bone marrow, lymph, and lymphatic system. Hematological malignancies may derive from either of the two major blood cell lineages: myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are from the lymphoid line, while acute and chronic myelogenous leukemia, myelodysplastic syndromes and myeloproliferative diseases are myeloid in origin.

A “chemotherapy agent,” “chemotherapeutic,” “anti-cancer agent” or the like, refer to molecules that are recognized to aid in the treatment of a cancer. Contemplated examples include the following molecules or derivatives such as temozolomide, carmustine, bevacizumab, procarbazine, lomustine, vincristine, gefitinib, erlotinib, cisplatin, carboplatin, oxaliplatin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin, mithramycin, vinblastine, vindesine, vinorelbine, paclitaxel, taxol, docetaxel, etoposide, teniposide, amsacrine, topotecan, camptothecin, bortezomib, anagrelide, tamoxifen, toremifene, raloxifene, droloxifene, iodoxyfene, fulvestrant, bicalutamide, flutamide, nilutamide, cyproterone, goserelin, leuprorelin, buserelin, megestrol, anastrozole, letrozole, vorozole, exemestane, finasteride, marimastat, trastuzumab, cetuximab, dasatinib, imatinib, combretastatin, thalidomide, azacitidine, azathioprine, capecitabine, chlorambucil, cyclophosphamide, cytarabine, daunorubicin, doxifluridine, epothilone, irinotecan, mechlorethamine, mercaptopurine, mitoxantrone, pemetrexed, tioguanine, valrubicin and/or lenalidomide or combinations thereof such as cyclophosphamide, methotrexate, 5-fluorouracil (CMF); doxorubicin, cyclophosphamide (AC); mustine, vincristine, procarbazine, prednisolone (MOPP); sdriamycin, bleomycin, vinblastine, dacarbazine (ABVD); cyclophosphamide, doxorubicin, vincristine, prednisolone (CHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

### CXCR4 Modulating Compounds

In certain embodiments, this disclosure relates to aminopiperidine amides derivatives, salts, and derivatives thereof that are CXCR4 modulators. In certain embodiments, the aminopiperidine amides are compounds have the following formula:

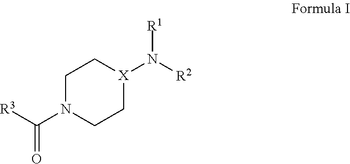

salts, derivatives, and prodrugs thereof wherein,

R¹ is 2-amino-5-guanidino-pentanoyl, 2-amino-2-phenylethanoyl, 2-amino-3-phenyl-propanoyl, 2-amino-3-(indol-3-yl)-propanoyl, 2-amino-3-(imidazol-4-yl)-propanoyl, 2-amino-4-(methylthio)butanoyl, pyrrolidine-2-carbonyl, 2-aminopropanoyl, 2-amino-3-mercaptopropanoyl, 2-aminoethanoyl, 2-amino-3-methylpentanoyl, 2,6-diaminohexanoyl, 2-amino methylpentanoyl, 2-amino-4-(methylthio)-butanoyl, 2-amino-3-carbamoyl-propanoyl, 2-amino-4-carbamoyl-butanoyl, 2-amino-3-hydroxy-propanoyl, 2-amino-3-hydroxy-butanyl, 2-amino methyl-butanoyl, 2-amino-3-(4-hydroxyphenyl)-propanoyl, alkyl, alkanoyl, carbocyclyl, benozyl, benzyl, aryl, or heterocyclyl, wherein R¹ is optionally substituted with one or more, the same or different, R¹⁰;

R² is hydrogen or alkyl; or

R¹ and R² and the attached nitrogen come together to form a heterocycle optionally substituted with one or more, the same or different, R¹⁰,

R³ is heterocyclylalkyl, alkyl, formyl, alkanoyl, alkoxycarbonyl, carbocyclyl, benozyl, benzyl, aryl, or heterocyclyl, wherein R³ is optionally substituted with one or more, the same or different, R¹⁰;

R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, carbocyclyl, benozyl, benzyl, aryl, or heterocyclyl, wherein R¹⁰ is optionally substituted with one or more, the same or different, R¹¹; and

R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydoxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methyl sulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, phenyl, benozyl, benzyl, carbocyclyl, aryl, or heterocyclyl.

In certain embodiments, R³ is heterocyclylalkyl, alkyl substituted with a heterocyclyl. In certain embodiments, the heterocyclyl is a bicyclic heterocycle having a five-membered ring and six-membered ring. In certain embodiments, R³ is alkyl substituted with pyrrolo[2,3-c]pyridinyl.

In certain embodiments, IV is alkanoyl substituted with one or more, the same or different, R¹⁰, and R³ is alkyl substituted with a heterocyclyl. In certain embodiments, the heterocyclyl is a bicyclic heterocycle having a five-membered ring and six-membered ring. In certain embodiments, R³ is alkyl substituted with pyrrolo[2,3-c]pyridinyl.

R¹ is 2-amino-5-guanidino-pentanoyl, 2-amino-2-phenylethanoyl, 2-amino-3-phenyl-propanoyl, 2-amino-3-(indol-3-yl)-propanoyl, 2-amino-3-(imidazol-4-yl)-propanoyl, 2-amino-4-(methylthio)butanoyl, pyrrolidine-2-carbonyl, 2-aminopropanoyl, 2-amino-3-mercaptopropanoyl, 2-aminoethanoyl, 2-amino-3-methylpentanoyl, 2,6-diaminohexanoyl, 2-amino-4-methylpentanoyl, 2-amino-4-(methylthio)-butanoyl, 2-amino-3-carbamoyl-propanoyl, 2-amino-4-carbamoyl-butanoyl, 2-amino-3-hydroxy-propanoyl, 2-amino-3-hydroxy-butanyl, 2-amino-3-methyl-butanoyl, or 2-amino-3-(4-hydroxyphenyl)-propanoyl and R³ is alkyl substituted with a heterocyclyl. In certain embodiments, the heterocyclyl is a bicyclic heterocycle having a five-membered ring and six-membered ring. In certain embodiments, R³ is alkyl substituted with pyrrolo[2,3-c]pyridinyl.

In certain embodiments, the compound is N-(1-(3-(pyrrolo[2,3-c]pyridin-1-yl)propanoyl)piperidin-4-yl)-2-amino-2-phenylacetamide (ZINC 72372983), salts, derivatives, and prodrugs thereof.

In certain embodiments, the compound is N-(1-(3-(pyrrolo[2,3-c]pyridin-1-yl)propanoyl)piperidin-4-yl)-2-amino-5-guanidinopentanamide (Z7R), salts, derivatives, and prodrugs thereof.

In certain embodiments, the compound is N-(1-(3-(pyrrolo[2,3-c]pyridin-1-yl)propanoyl)piperidin-4-yl)-2-amino-3-(1H-indol-3-yl)propanamide (Z7W), salts, derivatives, and prodrugs thereof.

In certain embodiments, the compound is N-(1-(3-(pyrrolo[2,3-c]pyridin-1-yl)propanoyl)piperidin-4-yl)-2,6-diaminohexanamide (Z7K), salts, derivatives, and prodrugs thereof.

In certain embodiments, the compound is N-(1-(3-(pyrrolo[2,3-c]pyridin-1-yl)propanoyl)piperidin-4-yl)-2-amino-3-(1H-imidazol-5-yl)propanamide (Z7H), salts, derivatives, and prodrugs thereof.

In certain embodiments, the compound is N-(1-(3-(pyrrolo[2,3-c]pyridin-1-yl)propanoyl)piperidin-4-yl)-2-amino-4-(methylthio)butanamide (Z7M), salts, derivatives, and prodrugs thereof.

In certain embodiments, the compound is N¹-(1-(3-(pyrrolo[2,3-c]pyridin yl)propanoyl)piperidin-4-yl)-2-aminosuccinamide (Z7N), salts, derivatives, and prodrugs thereof.

In certain embodiments, the compound is N¹-(1-(3-(pyrrolo[2,3-c]pyridin yl)propanoyl)piperidin-4-yl)-2-aminopentanediamide (Z7Q), salts, derivatives, and prodrugs thereof.

In certain embodiments, the compound is N-(1-(3-(pyrrolo[2,3-c]pyridin-1-yl)propanoyl)piperidin-4-yl)-2-amino-3-hydroxypropanamide (Z7S), salts, derivatives, and prodrugs thereof.

In certain embodiments, the compound is N-(1-(3-(pyrrolo[2,3-c]pyridin-1-yl)propanoyl)piperidin-4-yl)pyrrolidine-2-carboxamide (Z7P), salts, derivatives, and prodrugs thereof.

In certain embodiments, the compound is N-(1-(3-(pyrrolo[2,3-c]pyridin-1-yl)propanoyl)piperidin-4-yl)-2-amino-3-(4-hydroxyphenyl)propenamide (Z7Y), salts, derivatives, and prodrugs thereof.

In certain embodiments, the compound is N-(1-(3-(pyrrolo[2,3-c]pyridin-1-yl)propanoyl)piperidin-4-yl)-2-amino-3-hydroxybutanamide (Z7T), salts, derivatives, and prodrugs thereof.

In certain embodiments, a compound of formula I has formula IA

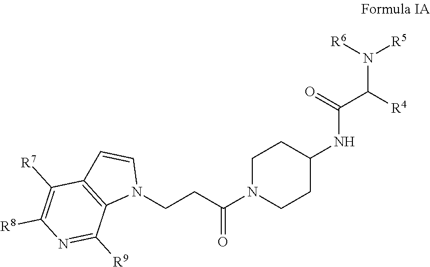

salts, derivatives, and prodrugs thereof wherein,

R⁴ is guanidinoalkyl, hydroxyalkyl, thioalkyl, aminoalkyl, heterocyclyalkyl, heteroarylalkyl, phenyl, or benzyl, wherein R⁴ is optionally substituted with one or more, the same or different, R¹⁰;

R⁵ is hydrogen or alkyl, wherein R⁵ is optionally substituted with one or more, the same or different, R¹⁰;

R⁶ is hydrogen or alkyl;

R⁷, R⁸, and R⁹ are each individually and independently hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, carbocyclyl, benozyl, benzyl, aryl, or heterocyclyl, wherein R⁷, R⁸, and R⁹ are optionally substituted with one or more, the same or different, R¹⁰;

R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, carbocyclyl, benozyl, benzyl, aryl, or heterocyclyl, wherein R¹⁰ is optionally substituted with one or more, the same or different, R¹¹; and

R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydoxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methyl sulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benozyl, benzyl, carbocyclyl, aryl, or heterocyclyl.

In certain embodiments, the aminopiperidine amides are compounds have the following formula:

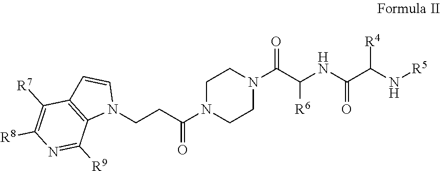

salts, derivatives, and prodrugs thereof wherein,

R⁴ is alkyl, guanidinoalkyl, hydroxyalkyl, thioalkyl, aminoalkyl, heterocyclyalkyl, heteroarylalkyl, phenyl, or benzyl, wherein R⁴ is optionally substituted with one or more, the same or different, R¹⁰;

R⁵ is hydrogen or alkyl, wherein R⁵ is optionally substituted with one or more, the same or different, R¹⁰;

R⁶ is hydrogen or alkyl;

R⁷, R⁸, and R⁹ are each individually and independently hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, carbocyclyl, benozyl, benzyl, aryl, or heterocyclyl, wherein R⁷, R⁸, and R⁹ are optionally substituted with one or more, the same or different, R¹⁰;

R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, carbocyclyl, benozyl, benzyl, aryl, or heterocyclyl, wherein R¹⁰ is optionally substituted with one or more, the same or different, R¹¹; and

R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydoxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methyl sulfinyl, ethylsulfinyl, mesyl, ethyl sulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benozyl, benzyl, carbocyclyl, aryl, or heterocyclyl.

In certain embodiments, the compound is N-(1-(4-(3-(1H-pyrrolo[2,3-c]pyridin-1-yl)propanoyl)piperazin-1-yl)-1-oxopropan-2-yl)-2-amino-2-phenylacetamide (ZINC 57773744).

In certain embodiments, the aminopiperidine amides are compounds have the following formula:

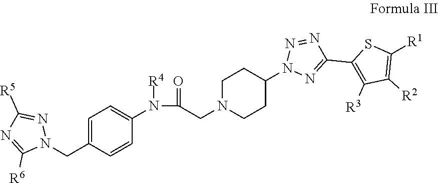

salts, derivatives, and prodrugs thereof wherein,

R¹, R², R³, R⁵, and R⁶ are each individually and independently hydrogen, halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydoxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methyl sulfamoyl, N-ethylsulfamoyl, N,N-dimethylsulfamoyl, N,N-diethylsulfamoyl, N-methyl-N-ethylsulfamoyl, benozyl, benzyl, carbocyclyl, aryl, or heterocyclyl; and

R⁴ is hydrogen or alkyl.

In certain embodiments, the compound is N-(4-((1H-1,2,4-triazol-1-yl)methyl)phenyl)-2-(4-(5-(thiophen-2-yl)-2H-tetrazol-2-yl)piperidin-1-yl)acetamide (Z08694519).

In certain embodiments, the aminopiperidine amides are compounds have the following formula:

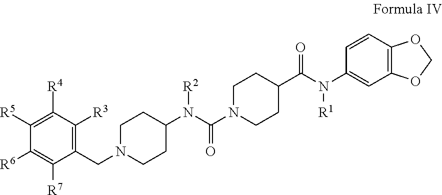

salts, derivatives, and prodrugs thereof wherein,

R¹ is hydrogen or alkyl;

R² is hydrogen or alkyl;

R³, R⁴, R⁵, R⁶ and R⁷ are each individually and independently hydrogen, halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydoxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methyl sulfinyl, ethyl sulfinyl, mesyl, ethyl sulfonyl, methoxycarbonyl, ethoxycarbonyl, isopropoxycarbonyl, tert-butoxycarbonyl, N-methyl sulfamoyl, N-ethylsulfamoyl, N,N-dimethylsulfamoyl, N,N-diethylsulfamoyl, N-methyl-N-ethylsulfamoyl, benozyl, benzyl, carbocyclyl, aryl, or heterocyclyl.

In certain embodiments, the compound is N⁴-(benzo[d][1,3]dioxol-5-yl)-N1-(1-benzylpiperidin-4-yl)piperidine-1,4-dicarboxamide (Z09388085).

### Methods of Use

In certain embodiments, this disclosure relates to methods of treating or preventing CXCR4 related diseases or conditions by administering an effective amount of a compound disclosed herein to a subject in need thereof.

In certain embodiments, the disclosure relates to methods of treating or preventing an inflammatory condition, cancer, viral infection, or enhancing the mobilization of hematopoietic stem and/or progenitor cells from the bone marrow to the peripheral blood comprising administering pharmaceutical composition comprising a compound disclosed herein to a subject in need thereof.

In certain embodiments, the disclosure relates to methods of treating or preventing an inflammatory condition comprising administering pharmaceutical composition comprising a compound disclosed herein to a subject in need thereof. In a particular embodiment, the compounds of the disclosure can be administered to a host at risk of, or suffering from, or diagnosed with an inflammatory condition. In one embodiment, the compounds are administered for the treatment or prophylaxis of an inflammatory disorder. In certain embodiments, the inflammatory disorder or condition is mediated by chemokines.

Generally, inflammatory disorders include, but are not limited to, respiratory disorders (including asthma, COPD, chronic bronchitis and cystic fibrosis); cardiovascular related disorders (including atherosclerosis, post-angioplasty, restenosis, coronary artery diseases and angina); inflammatory diseases of the joints (including rheumatoid and osteoarthritis); skin disorders (including dermatitis, eczematous dermatitis and psoriasis); post transplantation late and chronic solid organ rejection; multiple sclerosis; autoimmune conditions (including systemic lupus erythematosus, dermatomyositis, polymyositis, Sjogren's syndrome, polymyalgia rheumatica, temporal arteritis, Behcet's disease, Guillain Barre, Wegener's granulomatosus, polyarteritis nodosa); inflammatory neuropathies (including inflammatory polyneuropathies); vasculitis (including Churg-Strauss syndrome, Takayasu's arteritis); inflammatory disorders of adipose tissue; and proliferative disorders (including Kaposi's sarcoma and other proliferative disorders of smooth muscle cells).

In one embodiment, the compounds of the disclosure are administered to a patient suffering from a cardiovascular disorder related to inflammation. Cardiovascular inflammatory disorders include atherosclerosis, post-angioplasty, restenosis, coronary artery diseases, angina, and other cardiovascular diseases.

In certain embodiments the disorder is a non-cardiovascular inflammatory disorder such as rheumatoid and osteoarthritis, dermatitis, psoriasis, cystic fibrosis, post transplantation late and chronic solid organ rejection, eczematous dermatitis, Kaposi's sarcoma, or multiple sclerosis. In yet another embodiment, the compounds disclosed herein can be selected to treat anti-inflammatory conditions that are mediated by mononuclear leucocytes.

In certain embodiments, this disclosure contemplates that certain CXCR4 modulators cannot substantially mobilize stem cells improving the safety as anti-inflammatory agents without an undesirable effect of mobilizating stem cells.

In certain embodiments, the disclosure relates to methods of treating or preventing cancer comprising administering a compound disclosed herein to a subject in need thereof. In certain embodiments, the disclosure relates to methods of of treating or preventing cancer comprising administering pharmaceutical composition comprising a compound disclosed herein in combination with another anticancer agent to a subject in need thereof. In certain embodiments, the disclosure relates to methods of of treating or preventing cancer comprising administering pharmaceutical composition comprising a compound disclosed herein in combination with another anticancer agent to a subject in need thereof.

The compounds disclosed herein can be used to treat disorders of abnormal cell proliferation generally, examples of which include, but are not limited to, types of cancers and proliferative disorders listed below. Abnormal cellular proliferation, notably hyperproliferation, can occur as a result of a wide variety of factors, including genetic mutation, infection, exposure to toxins, autoimmune disorders, and benign or malignant tumor induction.

There are a number of skin disorders associated with cellular hyperproliferation. Psoriasis, for example, is a benign disease of human skin generally characterized by plaques covered by thickened scales. The disease is caused by increased proliferation of epidermal cells of unknown cause. In normal skin the time required for a cell to move from the basal layer to the upper granular layer is about five weeks. In psoriasis, this time is only 6 to 9 days, partially due to an increase in the number of proliferating cells and an increase in the proportion of cells which are dividing (G. Grove, Int. J. Dermatol. 18:111, 1979). Chronic eczema is also associated with significant hyperproliferation of the epidermis. Other diseases caused by hyperproliferation of skin cells include atopic dermatitis, lichen planus, warts, pemphigus vulgaris, actinic keratosis, basal cell carcinoma and squamous cell carcinoma.

Other hyperproliferative cell disorders include blood vessel proliferation disorders, fibrotic disorders, autoimmune disorders, graft-versus-host rejection, tumors and cancers.

Blood vessel proliferative disorders include angiogenic and vasculogenic disorders. Proliferation of smooth muscle cells in the course of development of plaques in vascular tissue cause, for example, restenosis, retinopathies and atherosclerosis. The advanced lesions of atherosclerosis result from an excessive inflammatory-proliferative response to an insult to the endothelium and smooth muscle of the artery wall (Ross, R. Nature, 1993, 362:801-809). Both cell migration and cell proliferation play a role in the formation of atherosclerotic lesions.

Fibrotic disorders are often due to the abnormal formation of an extracellular matrix. Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis.

Another disease with a proliferative component is rheumatoid arthritis. Rheumatoid arthritis is generally considered an autoimmune disease that is thought to be associated with activity of autoreactive T cells (See, e.g., Harris, E. D., Jr. (1990) The New England Journal of Medicine, 322:1277-1289), and to be caused by auto-antibodies produced against collagen and IgE.

Mesangial disorders are brought about by abnormal proliferation of mesangial cells. Mesangial hyperproliferative cell disorders include various human renal diseases, such as glomerulonephriti s, diabetic nephropathy, malignant nephrosclerosi s, thrombotic microangiopathy syndromes, transplant rejection, and glomerulopathies.

Other disorders that can include an abnormal cellular proliferative component include Behcet's syndrome, acute respiratory distress syndrome (ARDS), ischemic heart disease, post-dialysis syndrome, leukemia, acquired immune deficiency syndrome, vasculitis, lipid histiocytosis, septic shock and inflammation in general.

Examples of cancers or proliferative disorders which can be the primary tumor that is treated include but are not limited to neoplasms located in the: colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvis, skin, soft tissue, spleen, thorax, and urogenital tract.

In certain embodiments, the subject is diagnosed with acute childhood lymphoblastic leukemia; acute lymphoblastic leukemia, acute lymphocytic leukemia, acute myeloid leukemia, adrenocortical carcinoma, adult (primary) hepatocellular cancer, adult (primary) liver cancer, adult acute lymphocytic leukemia, adult acute myeloid leukemia, adult Hodgkin's disease, adult Hodgkin's lymphoma, adult lymphocytic leukemia, adult non-Hodgkin's lymphoma, adult primary liver cancer, adult soft tissue sarcoma, AIDS-related lymphoma, AIDS-related malignancies, anal cancer, astrocytoma, bile duct cancer, bladder cancer, bone cancer, brain stem glioma, brain tumors, breast cancer, cancer of the renal pelvis and ureter, central nervous system (primary) lymphoma, central nervous system lymphoma, cerebellar astrocytoma, cerebral astrocytoma, cervical cancer, childhood (primary) hepatocellular cancer, childhood (primary) liver cancer, childhood acute lymphoblastic leukemia, childhood acute myeloid leukemia, childhood brain stem glioma, childhood cerebellar astrocytoma, childhood cerebral astrocytoma, childhood extracranial germ cell tumors, childhood Hodgkin's disease, childhood Hodgkin's lymphoma, childhood hypothalanic and visual pathway glioma, childhood lymphoblastic leukemia, childhood medulloblastoma, childhood non-Hodgkin's lymphoma, childhood pineal and supratentorial primitive neuroectodermal tumors, childhood primary liver cancer, childhood rhabdomyosarcoma, childhood soft tissue sarcoma, childhood visual pathway and hypothalamic glioma, chronic lymphocytic leukemia, chronic myelogenous leukemia, colon cancer, cutaneous T-cell lymphoma, endocrine pancreas islet cell carcinoma, endometrial cancer, ependymoma, epithelial cancer, esophageal cancer, Ewing's sarcoma and related tumors, exocrine pancreatic cancer, extracranial germ cell tumor, extragonadal germ cell tumor, extrahepatie bile duct cancer, eye cancer, female Breast cancer, Gaucher's disease, gallbladder cancer, gastric cancer, gastrointestinal carcinoid tumor, gastrointestinal tumors, germ cell tumors, gestational trophoblastic tumor, hairy cell leukemia, head and neck cancer, hepatocellular cancer, Hodgkin's disease, Hodgkin's lymphoma, hypergammaglobulinemia, hypopharyngeal cancer, intestinal cancers, intraocular melanoma, islet cell carcinoma, islet cell pancreatic cancer, Kaposi's sarcoma, kidney cancer, laryngeal cancer, lip and oral cavity cancer, liver cancer, lung cancer, lympho proliferative disorders, macroglobulinemia, male breast cancer, malignant mesothelioma, malignant thymoma, medulloblastomia, melanoma, mesothelioma, metastatie occult primary squamous neck cancer, metastatie primary squamous neck cancer, metastatie squamous neck cancer, multiple myeloma, multiple myeloma/plasma cell neoplasm, myelodysplasia syndrome, myelogenous leukemia, myeloid leukemia, myeloproliferative disorders, nasal cavity and paranasal sinus cancer, nasopharyngeal cancer, neuroblastoma, non-Hodgkin's lymphoma during pregnancy, nonmelanoma skin cancer, non-small cell lung cancer, occult primary metastatie squamous neck cancer, oropharyngeal cancer, osteo/malignant fibrous sarcoma, osteosarcoma/malignant fibrous histiocytoma, osteosarcoma/malignant fibrous histiocytoma of bone, ovarian epithelial cancer, ovarian germ cell tumor, ovarian low malignant potential tumor, pancreatic cancer, paraproteinemias, parathyroid cancer, penile cancer, pheochromocytoma, pituitary tumor, plasma cell neoplasm/multiple myeloma, primary central nervous system lymphoma, primary liver cancer, prostate cancer, rectal cancer, renal cell cancer, renal pelvis and ureter cancer, retinoblastoma, rhabdomyosarcoma, salivary gland cancer, sarcoidosis sarcomas, sezary syndrome, skin cancer, small cell lung cancer, small Intestine cancer, soft tissue sarcoma, squamous neck cancer, stomach cancer, supratentorial primitive neuroectodermal and pineal tumors, T-cell lymphoma, testicular cancer, thymoma, thyroid cancer, transitional cell cancer of the renal pelvis and ureter, transitional renal pelvis and ureter cancer, trophoblastic tumors, ureter and renal pelvis cell cancer, urethial cancer, uterine cancer, uterine sarcoma, vaginal cancer, visual pathway and hypothalamic glioma, vulvar cancer, Waldenstrom's macroglobulinemia, Wilm's tumor, and any other hyperproliferative disease located in an organ system listed above.

In certain embodiments, the compound disclosed herein can be used to treat or prevent hyperplastic disorders including, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, foca epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia; leukemia (including acute leukemia (e.g., acute lymphocytic leukemia, acute myelocytic leukemia (including myeloblasts, promyelocyte, mylomonocytic, monocytic, and erythroleukemia)) and chronic leukemia (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and, carcinomas such as fibrosarcoma, myxosarcoma, fiposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, emangioblastoma, acoustic neuroma, oligodendrogliomia, menangioma, melanoma, neuroblastoma, and retinoblastoma.

In certain embodiments, a cancer treatment may be applied as a sole therapy or may involve, conventional surgery or radiotherapy or chemotherapy. Such chemotherapy may include with another agent such as abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof.

In a separate embodiment, the disclosure relates to a method for the treatment of, prevention of, or reduced severity of, age-related macular degeneration (ARMD) and other pathogenic states involving macular retinal pigment epithelial (RPE) cells by administering at least one compound described herein to a subject in need thereof.

CXCR4 plays a role in ocular diseases involving the retina such as age-related macular degeneration (ARMD). The retinal pigment epithelium has a major role in the physiological renewal of photoreceptor outer segments in the provision of a transport and storage system for nutrients essential to the photoreceptor layer. The retinal pigment epithelial (RPE) cells predominantly express CXCR4 receptors. (Crane, et al. (2000) J. Immunol. 165: 4372-4278). CXCR4 receptor expression on human retinal pigment epithelial cells from the blood-retina barrier leads to chemokine secretion and migration in response to stromal cell-derived factor Ia. J. Immunol. 200; 165: 4372-4278). The level of CXCR4 mRNA expression increases upon stimulation with IL-1β or TNFα (Dwinell, et al. (1999) Gastroenterology. 117: 359-367). RPE cells also migrated in response to SDF-1α indicating that SDF-1α/CXCR4 interactions may modulate the affects of chronic inflammation and subretinal neovascularization at the RPE site of the blood-retina barrier. (Crane U, Wallace C A, McKillop-Smith S, Forrester J V. CXCR4 receptor expression on human retinal pigment epithelial cells from the blood-retina barrier leads to chemokine secretion and migration in response to stromal cell-derived factor Ia. J. Immunol. 200; 165: 4372-4278).

Age-related macular degeneration is characterized by both primary and secondary damage of macular RPE cells. Early stages of ARMD are characterized by macular drusen, and irregular proliferation and atrophy of the RPE. The late stages of ARMD present with geographic RPE atrophy, RPE detachment and rupture, choroidal neovascularaization and fibrovascular disciform scarring. Common first symptoms include metamorphopisia and/or general central vision loss resulting in reading disability and difficulties in detecting faces. Late stages of ARMD cause central scomota, which is extremely disabling if occurrence is bilateral (Bressler and Bressler (1995) Ophthalmology. 1995; 102: 1206-1211).

In a separate embodiment, a method for the treatment of, prevention of, or reduced severity of inflammatory disease states, neovascularization, and wound healing including administering at least one compound described herein to a subject in need thereof. Vascular endothelial cells express a multitude of chemokine receptors, with CXCR4 being particularly prominent (Gupta, et al. (1998) J Biol Chem. 273: 4282; Volin, et al. (1998) Biochem Biophys Res Commnun. 242: 46).

Certain inflammatory chemokines can be induced during an immune response to promote cells of the immune system to a site of infection. Inflammatory chemokines function mainly as chemoattractants for leukocytes, recruiting monocytes, neutrophils and other effector cells from the blood to sites of infection or tissue damage. Certain inflammatory chemokines activate cells to initiate an immune response or promote wound healing. Responses to chemokines include increasing or decreasing expression of membrane proteins, proliferation, and secretion of effector molecules.

In certain embodiments, this disclosure relates to methods of enhancing the mobilization of hematopoietic stem and/or progenitor cells from the bone marrow to the peripheral blood in a cell donor. In certain embodiments, methods comprise the steps of: contacting hematopoietic stem and/or progenitor cells in a donor with an effective amount of a compound, wherein said compound is an modulator of CXCR4 thereby enhancing mobilization of hematopoietic stem and/or progenitor cells compared to the mobilization of hematopoietic stem and/or progenitor cells that have not been contacted with the compound, wherein the hematopoietic stem and/or progenitor cells are CD34+ and/or colony forming unit-granulocyte/macrophage (CFU-GM) cells.

In certain embodiments, the compound is administered to the donor for a period of time overlapping with co-treatment with at least one additional compound that enhances the mobilization of hematopoietic stem and/or progenitor cells. In certain embodiments, the additional compound that enhances the mobilization of hematopoietic stem and/or progenitor cells is selected from the group consisting of Granulocyte-Colony Stimulating Factor (G-CSF) and plerixafor. In certain embodiments, the compound is administered simultaneously in combination with G-CSF or plerixafor.

In certain embodiments, this disclosure relates to the enhancement of hematopoietic stem and progenitor cells harvesting and/or engraftment, some of these aspects include, but are not limited to, ex vivo survival, self-renewal and homing to appropriate marrow niches to increase the success rate for hematopoietic stem and progenitor cell therapy.

In certain embodiments, this disclosure relates to methods directed towards increasing the number of hematopoietic stem and progenitor cells with long-term repopulation capabilities harvested from a donor. Some of these methods comprise the steps of providing a pharmaceutically effective amount of the compound(s) to the donor prior to harvesting hematopoietic stem and progenitor cells from the donor's peripheral blood or bone marrow. In one embodiment, the compounds are used in combination with one or more clinically approved hematopoietic stem and progenitor cell mobilization agents, for example, Granulocyte-Colony Stimulating Factor (G-CSF), to increase the number of hematopoietic stem and progenitor cells that can be collected by apheresis for hematopoietic graft transplantation.

In certain embodiments, this disclosure relates to methods for enhancing a harvested hematopoietic stem and/or progenitor cells graft's long-term repopulation capability in a recipient. This may be of particular utility in situations in which the recipient has a compromised hematopoietic system. The method comprises the steps of a) harvesting a graft from a donor, wherein the donor has been treated with an effective amount of compound disclosed herein; b) contacting the graft with an effective amount of a compound disclosed herein ex vivo; and c) applying the treated graft to the recipient. In one embodiment, the method further comprises the step of supplying an effective amount of a compound disclosed herein to a transplant recipient in order to enhance the homing of the graft materials to their appropriate therapeutic niches.

In addition, the disclosure is directed to methods of treating animal subjects, in particular, veterinary and human subjects, to enhance or elevate the number of progenitor cells and/or stem cells. The progenitor and/or stem cells may be harvested and used in cell transplantation. In one embodiment, bone marrow progenitor and/or stem cells are mobilized for myocardial repair. Further, the disclosure is directed to methods of treating animal subjects, in particular, veterinary and human patients, who are defective in white blood cell count, or who would benefit from elevation of WBC levels using the compounds disclosed herein. Moreover, the disclosure is directed to methods of effecting regeneration of cardiac tissue in a subject in need of such regeneration using the disclosed compounds.

The compounds of the disclosure may be used for the treatment of diseases that are associated with immunosuppression such as individuals undergoing chemotherapy, radiation therapy, enhanced wound healing and burn treatment, therapy for autoimmune disease or other drug therapy (e.g., corticosteroid therapy) or combination of conventional drugs used in the treatment of autoimmune diseases and graft/transplantation rejection, which causes immunosuppression; immunosuppression due to congenital deficiency in receptor function or other causes; and infectious diseases, such as parasitic diseases, including but not limited to helminth infections, such as nematodes (round disclosure thus targets a broad spectrum of conditions for which elevation of progenitor cells and/or stem cells in a subject would be beneficial or, where harvesting of progenitor cells and/or stem cell for subsequent stem cell transplantation would be beneficial. In addition, the method of the disclosure targets a broad spectrum of conditions characterized by a deficiency in white blood cell count, or which would benefit from elevation of said WBC count.

In certain embodiments, the compounds described herein are useful for the treatment of microbial or viral infections where the virus utilized CXCR4 to infect cells. In certain embodiments, the disclosure relates to methods of treating or preventing a viral infection comprising administering a compound disclosed herein to a subject in need thereof. In certain embodiments, the disclosure relates to methods of treating or preventing a viral infection comprising administering pharmaceutical composition comprising a compound disclosed herein in combination with another antimicrobial, antibiotic, and/or antiviral agent to a subject in need thereof.

In one embodiment, the disclosure relates to a method of treating or preventing HIV infection or reduction of symptoms associated with AIDS is provided including administering a compound disclosed herein to a subject. In certain embodiments, the compound can be provided to a subject before treatment of infection with another compound. In a separate embodiment, the compound is provided to a patient that has been treated for HIV infection to reduce the likelihood of recurrence, or reduce mortality associated with AIDS related symptoms. In another embodiment, the compound is administered to a subject at high risk of suffering from HIV infections.

Subjects, including humans suffering from, or at risk for, HIV infection can be treated by administering an effective amount of the active compound or a pharmaceutically acceptable prodrug or salt thereof in the presence of a pharmaceutically acceptable carrier or diluent.

The administration can be prophylactically for the prevention of HIV infection or reduction of symptoms associated with AIDS. The active materials can be administered by any appropriate route, for example, orally, parenterally, intravenously, intradermally, subcutaneously, or topically, in liquid or solid form.

In a separate embodiment, a method for the treatment or prevention of HIV infection or reduction of symptoms associated with AIDS by administering a compound of the present disclosure, or a pharmaceutically acceptable salt, solvate, prodrug, or ester thereof to a subject in need of treatment is provided. The compounds of the disclosure, or a pharmaceutically acceptable salt, solvate, prodrug, or ester thereof can be administered to a subject in need thereof to reduce the severity of AIDS related disorders. In one embodiment of the disclosure, the subject is a human.

In a separate embodiment, a method for the treatment of, prevention of, or reduced severity of liver disease associated with viral infections including administering at least one compound described herein is provided.

Chronic hepatitis C virus (HCV) and hepatitis B virus (HBC) infection is accompanied by inflammation and fibrosis eventually leading to cirrhosis. A study testing the expression and function of CXCR4 on liver-infiltrating lymphocytes (LIL) revealed an important role for the CXCL12/CXCR4 pathway in recruitment and retention of immune cells in the liver during chronic HCV and HBV infection (Wald et al., (2004) European Journal of Immunology. 34(4): 1164-1174). High levels of CXCR4 and TGFβ have been detected in liver samples obtained from patients infected with HCV. (Mitra et al., (1999) Int. J. Oncol. 14: 917-925). In vitro, TGF-β has been shown to up-regulate the expression of CXCR4 on T cells and to increase their migration. The CD69/TGFβ/CXCR4 pathway may be involved in the retention of recently activated lymphocytes in the liver (Wald et al., European Journal of Immunology, 2004; 34(4): 1164-1174).

In another embodiment, the disclosure relates to a method of treating symptoms associated with other infections associated with chemokine receptor activation, for example, liver diseases associated with flavivirus or pestivirus infection, and in particular, HCV or HBV, by contacting a cell with a compound of the present disclosure, or a pharmaceutically acceptable salt, solvate, prodrug, or ester thereof. The cell can be in a subject animal, in particular in a human.

In certain embodiments, the disclosure relates to administering chemokine CXCR4 receptor modulators disclosed herein in combination with natural ligands of CXCR4. The natural ligands for the chemokine receptor CXCR4 (SDF-1) can act as potent inhibitors of infection by the human immunodeficiency virus type 1 (HIV-1) at the level of viral entry. Unlike antibody-mediated inhibition, chemokine-mediated inhibition is broadly effective. Different HIV-1 strains can utilize the same co-receptor(s) for viral entry and, therefore, can be blocked by the same chemokine(s). HIV-1 strains that are highly resistant to neutralization by V3-specific antibodies are sensitive to inhibition by chemokines. Therefore, the use of chemokine-modulators constitutes a therapeutic approach to prevent infection by HIV-1. Alkhatib et al., Science. 1996, 272: 1955-1988 and Challita-Eid et al., AIDS Research and Human Retroviruses, 1998, 14(18): 1617-1624.

In some embodiments, the disclosure relates to treating a viral infection by administering a CXCR4 modulator in combination with another, second antiviral agent. In specific embodiments, the compounds described herein are administered in combination or alternation with at least one compound that inhibits HIV entry into a cell through a mechanism not dependent on CXCR4, and in particular embodiments, are administered in combination or alternation with a compound that inhibits CCR5, gp120, gp41 or CD4 binding or activity. In some embodiments, such a compound is at least one of Maraviroc (Celsentri) or Enfuvirtide (Fuzeon). In yet further embodiments such compound is selected from TNX-355, PRO 250, BMS-488043, a theaflavin, Vicriviroc, Gruffithsin, DCM205, ESN196, TBR220, TMB355, Nifeviroc, BMS663068, CYT107, Sifuvirtide, AMD070, PF232798, SP01A.

In further embodiments, the subject is co-administered with abacavir, acyclovir, acyclovir, adefovir, amantadine, amprenavir, ampligen, arbidol, atazanavir, atripla, boceprevir, cidofovir, combivir, darunavir, delavirdine, didanosine, docosanol, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, methisazone, nelfinavir, nevirapine, nexavir, oseltamivir (Tamiflu), peginterferon alfa-2a, penciclovir, peramivir, pleconaril, podophyllotoxin, raltegravir, ribavirin, rimantadine, ritonavir, pyramidine, saquinavir, stavudine, tenofovir, tenofovir disoproxil, tipranavir, trifluridine, trizivir, tromantadine, truvada, valaciclovir (Valtrex), valganciclovir, vicriviroc, vidarabine, viramidine zalcitabine, zanamivir (Relenza), and/or zidovudine.

HIV is typically treated with a combination of antiviral agent, e.g., two nucleoside-analogue reverse transcription inhibitors and one non-nucleoside-analogue reverse transcription inhibitor or protease inhibitor. The three-drug combination is commonly known as a triple cocktail. In certain embodiments, the disclosure relates to treating a subject diagnosed with HIV by administering a chemokine CXCR4 receptor modulator disclosed herein in combination with two nucleoside-analogue reverse transcription inhibitors and/or one non-nucleoside-analogue reverse transcription inhibitor or protease inhibitor.

In certain embodiments, the disclosure relates to treating a subject by administering a chemokine CXCR4 receptor modulator disclosed herein, emtricitabine, tenofovir, and efavirenz. In certain embodiments, the disclosure relates to treating a subject by administering a chemokine CXCR4 receptor modulator disclosed herein, emtricitabine, tenofovir and raltegravir. In certain embodiments, the disclosure relates to treating a subject by administering a chemokine CXCR4 receptor modulator disclosed herein, emtricitabine, tenofovir, ritonavir and darunavir. In certain embodiments, the disclosure relates to treating a subject by administering a chemokine CXCR4 receptor modulator disclosed herein, emtricitabine, tenofovir, ritonavir and atazanavir.

### Pharmaceutical Compositions

In certain embodiments, the disclosure relates to pharmaceutical composition comprising a CXCR4 modulating compound disclosed herein or salt or derivative or prodrug thereof and a pharmaceutically acceptable excipient, diluent, or carrier. In certain embodiments, the pharmaceutical composition further comprises another active ingredient.

In certain embodiments, the disclosure relates to pharmaceutical compositions comprising a CXCR4 modulating compound disclosed herein with another active ingredient.

In certain embodiments, the pharmaceutical composition is in the form of a tablet, pill, capsule, gel, gel capsule, or cream. In certain embodiments, the pharmaceutical composition is in the form of a sterilized pH buffered aqueous salt solution or a saline phosphate buffer between a pH of 6 to 8, optionally comprising a saccharide or polysaccharide.

In certain embodiments, the pharmaceutical composition is in solid form surrounded by an enteric coating. In certain embodiments, the enteric coatings comprises methyl acrylate-methacrylic acid copolymers, cellulose acetate phthalate (CAP), cellulose acetate succinate, hypromellose (hydroxypropyl methylcellulose), hypromellose phthalate (hydroxypropyl methyl cellulose phthalate), hypromellose acetate succinate (hydroxypropyl methyl cellulose acetate succinate), diethyl phthalate, polyvinyl acetate phthalate (PVAP), methyl methacrylate-methacrylic acid copolymers, or combinations thereof.

In certain embodiments, the pharmaceutically acceptable excipient is selected from lactose, sucrose, mannitol, triethyl citrate, dextrose, cellulose, microcrystalline cellulose, methyl cellulose, ethyl cellulose, hydroxyl propyl cellulose, hydroxypropyl methylcellulose, carboxymethylcellulose, croscarmellose sodium, polyvinyl N-pyrrolidone (crospovidone), ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, acacia gum, esters, or salts thereof.

Pharmaceutical compositions disclosed herein may be in the form of pharmaceutically acceptable salts, as generally described below. Some preferred, but non-limiting examples of suitable pharmaceutically acceptable organic and/or inorganic acids are hydrochloric acid, hydrobromic acid, sulfuric acid, nitric acid, acetic acid and citric acid.

When the compounds of the disclosure contain an acidic group as well as a basic group, the compounds of the disclosure may also form internal salts, and such compounds are within the scope of the disclosure. When a compound contains a hydrogen-donating heteroatom (e.g. NH), salts are contemplated to covers isomers formed by transfer of said hydrogen atom to a basic group or atom within the molecule.

Pharmaceutically acceptable salts of the compounds include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds described herein may be administered in the form of prodrugs. A prodrug can include a covalently bonded carrier which releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a mammalian subject, cleaves to form a free hydroxyl group. Examples of prodrugs include, but are not limited to, acetate, formate and benzoate derivatives of alcohol functional groups in the compounds. Methods of structuring a compound as prodrugs can be found in the book of Testa and Mayer, Hydrolysis in Drug and Prodrug Metabolism, Wiley (2006). Typical prodrugs form the active metabolite by transformation of the prodrug by hydrolytic enzymes, the hydrolysis of amide, lactams, peptides, carboxylic acid esters, epoxides or the cleavage of esters of inorganic acids.

Pharmaceutical compositions for use in the present disclosure typically comprise an effective amount of a compound and a suitable pharmaceutical acceptable carrier. The preparations may be prepared in a manner known per se, which usually involves mixing the at least one compound according to the disclosure with the one or more pharmaceutically acceptable carriers, and, if desired, in combination with other pharmaceutical active compounds, when necessary under aseptic conditions. Reference is again made to U.S. Pat. Nos. 6,372,778, 6,369,086, 6,369,087 and 6,372,733 and the further references mentioned above, as well as to the standard handbooks, such as the latest edition of Remington's Pharmaceutical Sciences.

Generally, for pharmaceutical use, the compounds may be formulated as a pharmaceutical preparation comprising at least one compound and at least one pharmaceutically acceptable carrier, diluent or excipient and/or adjuvant, and optionally one or more further pharmaceutically active compounds.

The pharmaceutical preparations of the disclosure are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the disclosure, e.g. about 10, 25, 50, 100, 200, 300 or 400 mg per unit dosage.

The compounds can be administered by a variety of routes including the oral, ocular, rectal, transdermal, subcutaneous, intravenous, intramuscular or intranasal routes, depending mainly on the specific preparation used. The compound will generally be administered in an “effective amount”, by which is meant any amount of a compound that, upon suitable administration, is sufficient to achieve the desired therapeutic or prophylactic effect in the subject to which it is administered. Usually, depending on the condition to be prevented or treated and the route of administration, such an effective amount will usually be between 0.01 to 1000 mg per kilogram body weight of the patient per day, more often between 0.1 and 500 mg, such as between 1 and 250 mg, for example about 5, 10, 20, 50, 100, 150, 200 or 250 mg, per kilogram body weight of the patient per day, which may be administered as a single daily dose, divided over one or more daily doses. The amount(s) to be administered, the route of administration and the further treatment regimen may be determined by the treating clinician, depending on factors such as the age, gender and general condition of the patient and the nature and severity of the disease/symptoms to be treated. Reference is again made to U.S. Pat. Nos. 6,372,778; 6,369,086; 6,369,087; and 6,372,733 and the further references mentioned above, as well as to the standard handbooks, such as the latest edition of Remington's Pharmaceutical Sciences.

Depending upon the manner of introduction, the compounds described herein may be formulated in a variety of ways. Formulations containing one or more compounds can be prepared in various pharmaceutical forms, such as granules, tablets, capsules, suppositories, powders, controlled release formulations, suspensions, emulsions, creams, gels, ointments, salves, lotions, or aerosols and the like. Preferably, these formulations are employed in solid dosage forms suitable for simple, and preferably oral, administration of precise dosages. Solid dosage forms for oral administration include, but are not limited to, tablets, soft or hard gelatin or non-gelatin capsules, and caplets. However, liquid dosage forms, such as solutions, syrups, suspension, shakes, etc. can also be utilized. In another embodiment, the formulation is administered topically. Suitable topical formulations include, but are not limited to, lotions, ointments, creams, and gels. In a preferred embodiment, the topical formulation is a gel. In another embodiment, the formulation is administered intranasally.

Formulations containing one or more of the compounds described herein may be prepared using a pharmaceutically acceptable carrier composed of materials that are considered safe and effective and may be administered to an individual without causing undesirable biological side effects or unwanted interactions. The carrier is all components present in the pharmaceutical formulation other than the active ingredient or ingredients. As generally used herein “carrier” includes, but is not limited to, diluents, binders, lubricants, disintegrators, fillers, pH modifying agents, preservatives, antioxidants, solubility enhancers, and coating compositions.

Carrier also includes all components of the coating composition which may include plasticizers, pigments, colorants, stabilizing agents, and glidants. Delayed release, extended release, and/or pulsatile release dosage formulations may be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-beta-alanine, sodium N-lauryl-beta-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

The concentration of the compound(s) to carrier and/or other substances may vary from about 0.5 to about 100 wt % (weight percent). For oral use, the pharmaceutical formulation will generally contain from about 5 to about 100% by weight of the active material. For other uses, the pharmaceutical formulation will generally have from about 0.5 to about 50 wt. % of the active material.

The compositions described herein can be formulation for modified or controlled release. Examples of controlled release dosage forms include extended release dosage forms, delayed release dosage forms, pulsatile release dosage forms, and combinations thereof.

The extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

### EXPERIMENTAL

### Experiments with Aminopiperidinyl Amide CXCR4 Modulators

CXCR4 has rapidly emerged as an attractive therapeutic target because it is involved in the pathogenesis of various diseases including HIV-infection, rheumatoid arthritis, WHIM syndrome, inflammatory bowel disease, pulmonary fibrosis, lupus, and metastatic cancer through the interaction with HIV glycoprotein 120 or endogenous chemokine ligand 12 (CXCL12) that is also known as stromal-derived-factor-1 (SDF-1). A chemical database was screened for CXCR4 modulators through a series of in silico guided processes. After evaluating the screened compounds for their in vitro binding affinities to CXCR4 and inhibitory activities against the chemoattractant CXCL12, compound (ZINC 72372983) showed 100 nM affinity and inhibited 69% chemotaxis at the same concentration (100 nM). A derivative compound (Z7R) was synthesized and showed nanomolar affinity (IC₅₀=1.25 nM) with improved chemotaxis inhibition (78.5%). Z7R displays promising anti-inflammatory activity (50%) in a mouse edema model by blocking CXCR4-expressed leukocytes in an immunohistochemistry study.

### Synthetic Methods

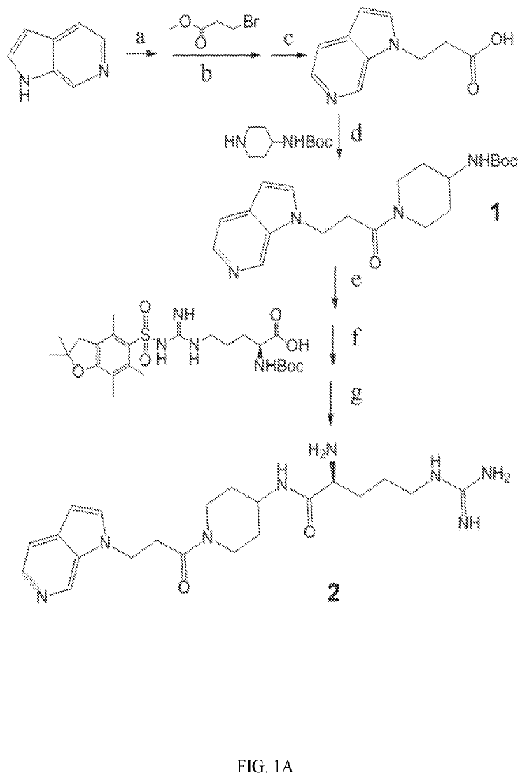

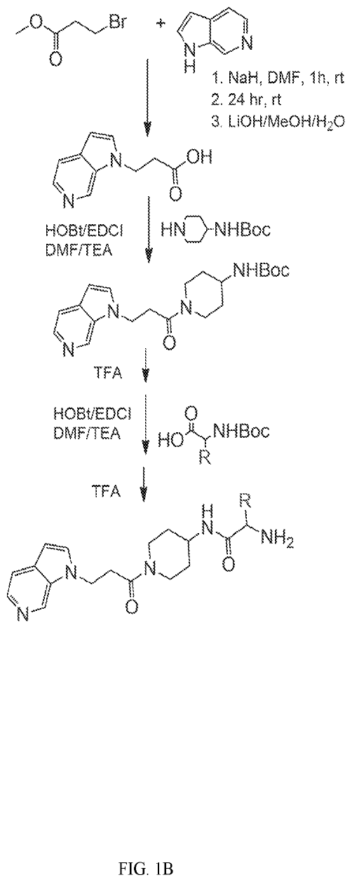

The synthesis of certain compounds are illustrated in FIGS. 1A-B. Additional compounds of this disclosure can be prepared in the same manner by substituting appropriate starting materials.

### Tert-butyl(1-(3-(1H-pyrrolo[2,3-c]pyridin-1-yl)propanoyl)piperidin-4-yl)carbamate (Compound 1)

A 100 mL round bottom flask fitted with magnetic stirrer was charged with 10 mL of DMF. To the stirred solvent was added 6-azaindole (1.01 g, 8.53 mmole) followed by sodium hydride (400 mg, 10.2 mmole) and stirred at RT for 1 h. Then, methyl-3-bromo-propioate (2.13 g, 12.79 mmole) was added and stirred at RT for 24 h. After completion of reaction, the reaction mass was diluted with 30 mL of water and extracted with chloroform (3×100 mL). The combined organic layer was washed with brine, dried over Na₂SO₄, and then concentrated under reduced pressure. The crude product was purified by flash column chromatography, eluting with DCM/methanol (20:1, v/v). The purified compound was obtained as colorless oil. (958 mg, yield 55%). ¹H NMR (400 MHz, CD₃Cl): δ 8.75 (1H, m), 8.21 (1H, d), 7.47 (1H, d), 7.26 (1H, d), 6.44 (1H, d), 4.51 (2H, t), 3.62 (3H, s), 2.84 (3H, t) ppm.

The purified compound (methyl 3-(1H-pyrrolo[2,3-c]pyridin-1-yl)propanoate, 958 mg, 4.70 mmole) was dissolved in methanol (10 mL) and water (10 mL), then was added lithium hydroxide (LiOH.H₂O, 1.0 g) and stirred overnight at RT. The reaction solvent was removed by rotary evaporator and dried reaction mixture was dissolved in DMF 20 mL, then was added t-butyl piperidin-4-ylcarbamate (939 mg, 4.70 mmole), N-(3-dimethylaminopropyl)-N′-ethyl carbodiimide hydrochloride (EDCI, 1.35 g, 7.04 mmole), 1-hydroxybenzotriazole (HOBt, 7.04 mmole), and triethylamine (1.4 mL). The reaction mixture was stirred overnight at RT. After the reaction was completed and diluted with water (5 mL), then extracted with ethyl acetate (3×100 mL), washed with water, brine, and dried over MgSO₄. The extract was concentrated, and crude mixture was purified by flash column chromatography, eluting with DCM/methanol (10:1, v/v). The purified compound was obtained as a white powder. (1.49 mg, yield 85%). ¹H NMR (400 MHz, CD₃Cl): δ 8.76 (1H, s), 8.18 (1H, d), 7.50 (1H, d), 7.35 (1H, d), 6.45 (1H, d), 4.58 (2H, m), 4.45 (2H, m), 3.54 (2H, d), 2.81 (4H, m), 2.65 (1H, m), 1.80 (2H, m), 1.39 (9H, s) ppm.

### Synthesis of N-(1-(3-(1H-pyrrolo[2,3-c]pyridin-1-yl)propanoyl)piperidin-4-yl)-2-amino-5-guanidinopentanamide (Z7R)

Compound 1 (1.49 mg, 4.00 mmol) was deprotected by stirring in cocktail solution composed of TFA (13 eqv.) and DCM (32 eqv.) in the presence of triisopropylsilane (TIPS, 2.5 eqv.) at RT with the exclusion of moisture. The deprotection was monitored by TLC. After completion of deprotection, the reaction mixture was dried under reduced pressure. The dried mixture was re-dissolved in DMF, then was added 2.11 mg (1 eqv.) of N-alpha-Boc-N-omega-(2,2,4,6,7-pentamethyl-2,3-dihydrobenzo[b]furan-5-ylsulfonyl)-arginine), N-(3-dimethylamino propyl)-N′-ethylcarbodiimide hydrochloride (EDCI, 1.5 eqv.), 1-hydroxybenzotriazole (HOBt, 1.5 eqv.), and triethylamine (4 eqv.). The reaction mixture was stirred overnight at RT. After the reaction was completed, diluted with water (5 mL) and extracted with ethyl acetate (3×100 mL), then washed with water, brine, and dried over MgSO₄ and concentrated. The extract was concentrated, and crude mixture was purified by flash column chromatography, eluting with DCM/methanol (10:1, v/v). The purified compound was obtained as yellow oil. (2.19 mg, yield 70%). The purified compound was deprotected by stirring in the cocktail solution (90% TFA, 5% TIPS, 5% water) and dried in vacuo. The crude mixture was dissolved in acetonitrile and precipitated with cold ether. The final compound was obtained as white crystalline powder. ¹H NMR (400 MHz, D20): δ 9.03 (1H, s), 8.11 (1H, d), 8.02 (2H, m), 6.90 (1H, s), 4.68 (2H, t), 4.12 (1H, br), 3.99 (1H, t), 3.90 (1H, m), 3.78 (2H, m), 3.73˜3.68 (2H, m), 3.21˜3.18 (4H, m), 3.01˜2.98 (3H, m), 2.73 (1H, m), 1.98˜1.82 (6H, m), 1.76˜1.67 (6H, m), 1.18 (2H, m). ¹³C NMR (400 MHz, D₂O): δ 170.6, 168.5, 156.6, 141.6, 138.2, 131.3, 127.5, 125.5, 117.3, 103.6, 52.7, 52.6, 46.6, 43.1, 40.1, 32.8, 27.8, 26.9, 23.6, 23.3. HRMS (ESI-TOF) m/z: [M+H]+ Calcd. for C₂₁H₃₃N₈O₂ 429.26; Found 429.27.

### In Vitro Assay

After computer evaluation of chemical libraries, seven compounds were tested by in vitro assays. The first binding assay quantified the affinity of compounds to the receptor by measuring the fluorescence of CXCR4-targeted peptidomimetic conjugate (rhodamine Red X-TN14003) after treating CXCR4-positive with each compound, which presents a lower fluorescent intensity for the compound with higher affinity. Among the identified candidates, 4 compounds showed a binding affinity (effective concentration: the concentration at which the compound blocks more than 50% of rhodamine Red X-TN14003, EC) less than 100 nM in our assay. As a secondary orthogonal functional assay, Matrigel invasion assays were performed which measures the inhibitory efficacies of compounds against the chemotaxis between CXCR4-positive cells and the chemokine, CXCL12. Through these consecutive assays, ZINC72372983 was identified as a compound which showed 100 nM affinity and 69% chemotaxis inhibition.

### In Vitro Binding Assay

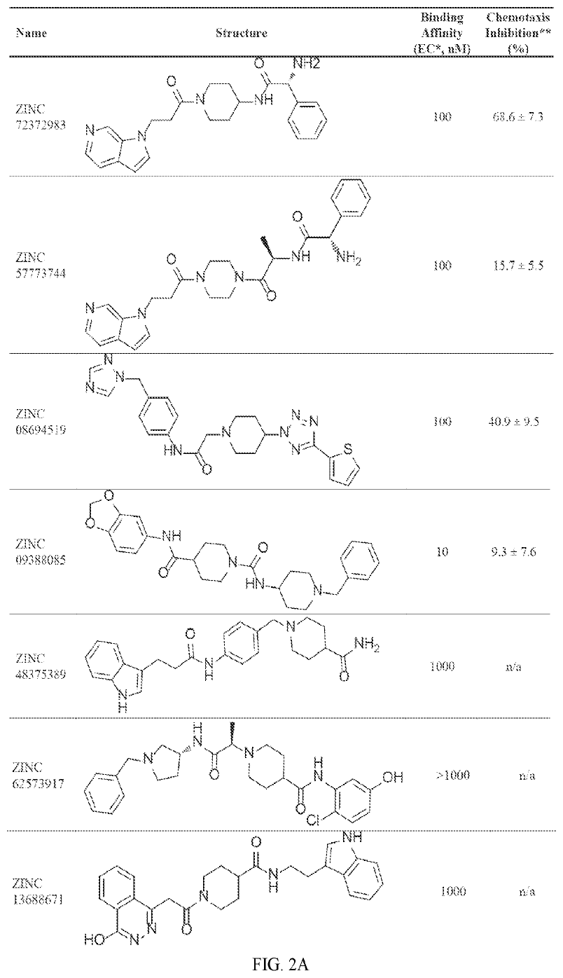

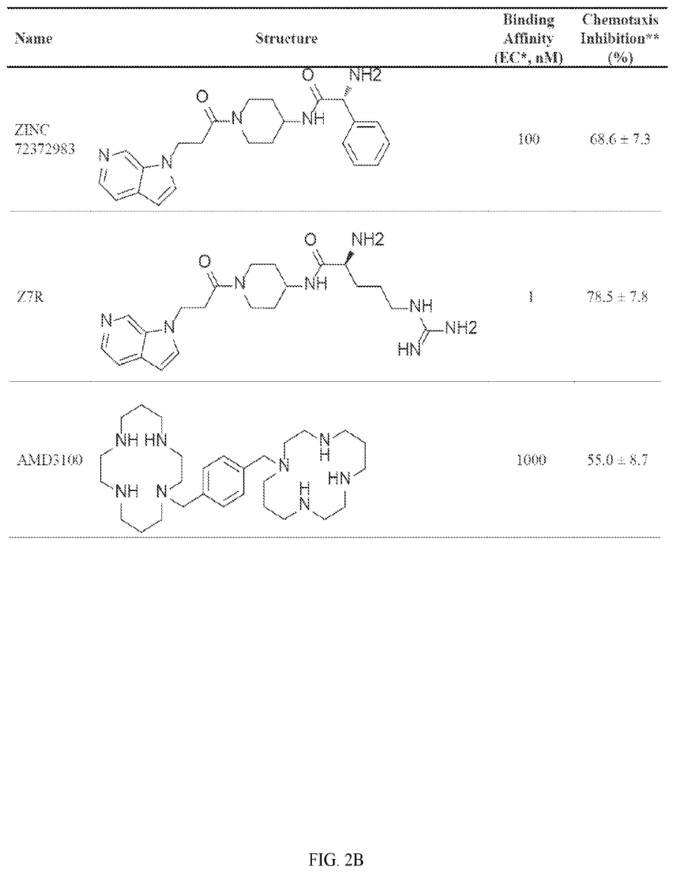

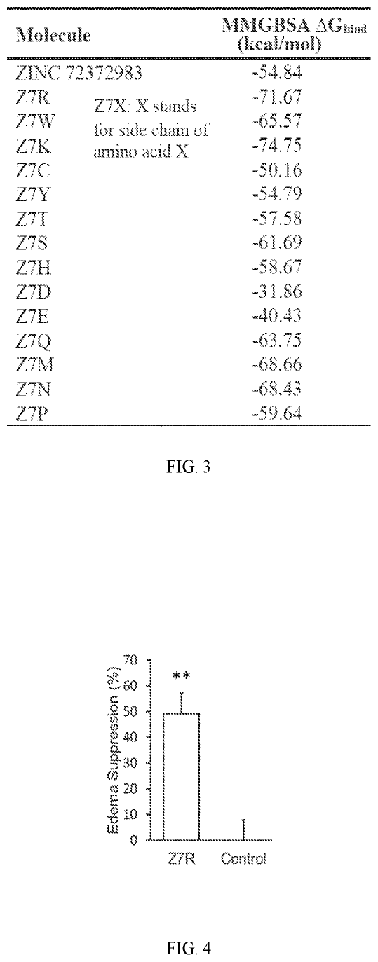

MDA-MB-231 cells were cultured in an eight-well slide chamber for two days. The cells were pre-incubated with the compounds for 15 min, and then fixed with 4% formaldehyde. The fixed cells were subsequently incubated for 45 min with biotinylated-TN14003 (CXCR4-targeted peptidomimetic peptide). Then, cells were incubated for 30 min in streptavidin—rhodamine at a 1:150 dilution (Jackson Immuno Research Laboratories, West Grove, Pa., USA) after washing three times with PBS. The slides were washed with PBS and mounted in an anti-fade mounting solution (Molecular Probes, Eugene, Oreg., USA). The pictures of stained cells for each treatment were taken on a Nikon Eclipse E800 microscope. Pictures were analyzed quantitatively with ImageJ. EC (effective concentration) is determined for the concentration at with the compound blocks >50% of CXCR4-targeted TN14003 conjugate using 1, 10, 100, and 1000 nM of compounds. IC₅₀ value of the hit compound was fitted with GraphPad Prism 5. Results when testing certain compounds are shown in FIGS. 2A and 2B.

### In Vitro Matrigel Invasion Assay

Matrigel invasion chambers were used for invasion assays. MDA-MB-231 cells were cultured on a layer of Matrigel in the upper chamber with compounds at 100 nM, while 200 ng/ml of CXCL12 was added in the lower chamber as a chemoattractant. The Matrigel invasion chamber was incubated for 22 h in a humidified tissue culture incubator. After non-invading cells were removed from the top of the Matrigel with a cotton-tipped swab. Invading cells at the bottom of the Matrigel were fixed in methanol and stained with H&E. The percentage of invading cells was determined by counting the H&E-stained cells. Results when testing certain compounds are shown in FIGS. 2A and 2B.

### In Vivo Anti-Inflammation Study Using Mouse Paw Edema Model

Acute inflammation was induced by subcutaneous injection of 504, of λ-carrageenan (1% w/v in saline) into the left hind paws of male nude mice (6 weeks, 20 g, Jackson Laboratory, Strain JAZ 007850 J:NU); the other hind paw was used as the non-inflammation control. The compounds were administered intraperitoneally (i.p. injection), 30 min after carrageenan challenge with the dose of 10 mg/kg using the formulation of 10% DMSO and 40% (2-hydroxypropyl)-β-cyclodextrin (CD) in PBS, and then once a day following the initial dose. The mice were sacrificed 74 h after inflammation was induced and 2 h after the last injection of the compounds. The hind paws of the mice were photographed, the thicknesses of the paws were measured by a caliper, and the weights of the paws were measured by a balance after transecting at the level of the malleoli. To quantify the edema, the measured values of the untreated group were subtracted from the treated group. The inflammation suppression percentage was calculated by comparing the compound-treated groups to the control group (n=5). Fixed paw tissue was sliced and stained with H&E for histology study.

To validate the physiological effects of compound Z7R which showed promising results in in vitro assays, carrageenan-induced paw edema (CIPE) assays were conducted using our mouse model. Z7R-treated mice (n=5) showed significant reductions of the edema weights (˜50%) in the lesion, compared with untreated mice (FIG. 4).

### Immunohistology Study

The avidin-biotin complex method was used to detect the proteins CXCR4 with anti-CXCR4 antibody (R&D System) at dilution 1:500. Formalin-fixed and paraffin-embedded tissues were deparaffinized and subsequently microwaved in 10 mM of sodium citrate buffer (pH 6.0) to retrieve antigens. After pre-incubation with hydrogen peroxide, avidin/biotin blocking kit (Invitrogen Co, CA), and horse serum (Vector Laboratories), the primary antibodies were applied overnight at 4° C. temperature. After incubation with the secondary antibody (rabbit anti-mouse biotinylated; Invitrogen Co, CA), the avidin-biotin complex was added and the enzyme activity was visualized with diaminobenzidine (ABC kit, Vector laboratories). Counterstaining was performed with hematoxylin.

Immunohistochemistry study revealed a large number of CXCR4-expressing infiltrating cells in the carrageenan-induced inflammatory tissue, compared with a reduced presence of CXCR4s which are mainly expressed in the epidermis of Z7R-treated mouse tissue. The immunohistochemistry study suggests that Z7R can suppress the inflammation by inhibiting chemotaxis of CXCR4-expressing leukocytes.

What is claimed is:

1. A compound of Formula I 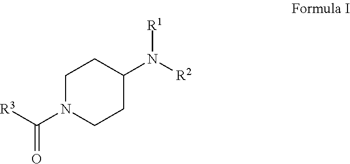
salts, and prodrugs thereof wherein,
R¹ is 2-amino-5-guanidino-pentanoyl, 2-amino-2-phenylethanoyl, 2-amino-3-phenyl-propanoyl, 2-amino-3-(1H-indol-3-yl)-propanoyl, 2-amino-3-(1H-imidazol-4-yl)-propanoyl, 2-amino-4-(methylthio)butanoyl, pyrrolidine-2-carbonyl, 2-aminopropanoyl, 2-amino-3-mercaptopropanoyl, 2-aminoethanoyl, 2-amino-3-methylpentanoyl, 2,6-diaminohexanoyl, 2-amino-4-methylpentanoyl, 2-amino-4-(methylthio)-butanoyl, 2-amino-3-carbamoyl-propanoyl, 2-amino-4-carbamoyl-butanoyl, 2-amino-3-hydroxy-propanoyl, 2-amino-3-hydroxy-butanyl, 2-amino-3-methyl-butanoyl, 2-amino-3-(4-hydroxyphenyl)-propanoyl, alkyl, alkanoyl, carbocyclyl, benzoyl, benzyl, aryl, or heterocyclyl, wherein R¹ is optionally substituted with one or more, the same or different, R¹⁰;
R² is hydrogen or alkyl; or
R¹ and R² and the attached nitrogen come together to form a heterocycle optionally substituted with one or more, the same or different, R¹⁰,
R³ is heterocyclylalkyl, alkyl, formyl, alkanoyl, alkoxycarbonyl, carbocyclyl, benzoyl, benzyl, aryl, or heterocyclyl, wherein R³ is optionally substituted with one or more, the same or different, R¹⁰;
R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkyl sulfonyl, aryl sulfonyl, carbocyclyl, benzoyl, benzyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydoxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methyl sulfinyl, ethylsulfinyl, mesyl, ethyl sulfonyl, methoxycarbonyl, ethoxycarbonyl, isopropoxycarbonyl, tert-butoxycarbonyl, N-methyl sulfamoyl, N-ethyl sulfamoyl, N,N-dimethylsulfamoyl, N,N-di ethyl sulfamoyl, N-methyl-N-ethyl sulfamoyl, phenyl, benzoyl, benzyl, carbocyclyl, aryl, or heterocyclyl.

2. The compound of claim 1 wherein R³ is alkyl substituted with a heterocyclyl.

3. The compound of claim 1 wherein the heterocyclyl is a bicyclic heterocycle having a five-membered ring and six-membered ring.

4. The compound of claim 1 wherein R¹ is alkanoyl substituted with one or more, the same or different, R¹⁰.

5. The compound of claim 1 which is the compound N-(1-(3-(1H-pyrrolo[2,3-c]pyridin-1-yl)propanoyl)piperidin-4-yl)-2-amino-5-guanidinopentanamide (Z7R).

6. The compound of claim 1 selected from:
N-(1-(3-(1H-pyrrolo[2,3-c]pyridin-1-yl)propanoyl)piperidin-4-yl)-2-amino-2-phenylacetamide (ZINC 72372983);
N-(1-(3-(pyrrolo[2,3-c]pyridin-1-yl)propanoyl)piperidin-4-yl)-2-amino-3-(1H-indol-3-yl)propanamide (Z7W);
N-(1-(3-(pyrrolo[2,3-c]pyridin-1-yl)propanoyl)piperidin-4-yl)-2,6-diaminohexanamide (Z7K);
N-(1-(3-(pyrrolo[2,3-c]pyridin-1-yl)propanoyl)piperidin-4-yl)-2-amino-3-(1H-imidazol-5-yl)propanamide (Z7H);
N-(1-(3-(pyrrolo[2,3-c]pyridin-1-yl)propanoyl)piperidin-4-yl)-2-amino-4-(methylthio)butanamide (Z7M);
N¹-(1-(3-(pyrrolo[2,3-c]pyridin-1-yl)propanoyl)piperidin-4-yl)-2-aminosuccinamide (Z7N);
N¹-(1-(3-(pyrrolo[2,3-c]pyridin-1-yl)propanoyl)piperidin-4-yl)-2-aminopentanediamide (Z7Q);
N-(1-(3-(pyrrolo[2,3-c]pyridin-1-yl)propanoyl)piperidin-4-yl)-2-amino-3-hydroxypropanamide (Z7S);
N-(1-(3-(pyrrolo[2,3-c]pyridin-1-yl)propanoyl)piperidin-4-yl)pyrrolidine-2-carboxamide (Z7P);
N-(1-(3-(pyrrolo[2,3-c]pyridin-1-yl)propanoyl)piperidin-4-yl)-2-amino-3-(4-hydroxyphenyl)propenamide (Z7Y); and
N-(1-(3-(pyrrolo[2,3-c]pyridin-1-yl)propanoyl)piperidin-4-yl)-2-amino-3-hydroxybutanamide (Z7T).

7. A compound having formula IA 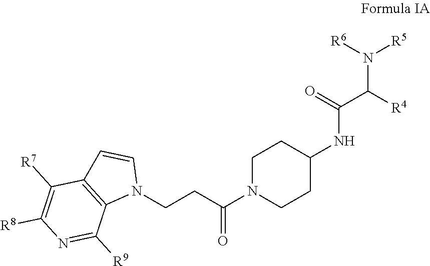
salts, and prodrugs thereof wherein,
R⁴ is guanidinoalkyl, hydroxyalkyl, thioalkyl, aminoalkyl, phenyl, or benzyl, wherein R⁴ is optionally substituted with one or more, the same or different, R¹⁰;
R⁵ is hydrogen or alkyl, wherein R⁵ is optionally substituted with one or more, the same or different, R¹⁰;
R⁵ is hydrogen or alkyl, wherein R⁵ is optionally substituted with one or more, the same or different, R¹⁰;
R⁶ is hydrogen or alkyl;
R⁷, R⁸, and R⁹ are each individually and independently hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, carbocyclyl, benzoyl, benzyl, aryl, or heterocyclyl, wherein R⁷, R⁸, and R⁹ are optionally substituted with one or more, the same or different, R¹⁰;
R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkyl sulfinyl, alkyl sulfonyl, aryl sulfonyl, carbocyclyl, benzoyl, benzyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydoxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methyl sulfinyl, ethylsulfinyl, mesyl, ethyl sulfonyl, methoxycarbonyl, ethoxycarbonyl, isopropoxycarbonyl, tert-butoxycarbonyl, N-methyl sulfamoyl, N-ethyl sulfamoyl, N,N-dimethylsulfamoyl, N,N-diethylsulfamoyl, N-methyl-N-ethylsulfamoyl, benzoyl, benzyl, carbocyclyl, aryl, or heterocyclyl.

8. A pharmaceutical comprising a compound of claim 1 or salt or prodrug thereof and a pharmaceutically acceptable excipient, diluent, or carrier.

9. The pharmaceutical composition of claim 8 further comprising another active ingredient.

10. A method of treating or preventing an inflammatory condition comprising administering pharmaceutical composition comprising a compound of claim 1 in combination with another active ingredient to a subject in need thereof.

11. A method of treating or preventing radiation-induced pulmonary fibrosis comprising administering pharmaceutical composition comprising a compound of claim 1 to a subject in need thereof.

12. A method of treating or preventing cancer comprising administering pharmaceutical composition comprising a compound of claim 1 to a subject in need thereof.

13. A method of treating or preventing a viral infection comprising administering pharmaceutical composition comprising a compound of claim 1 to a subject in need thereof.

14. A method of enhancing the mobilization of hematopoietic stem and/or progenitor cells from the bone marrow to the peripheral blood in a cell donor comprising administering an effective amount of a compound of claim 1 to the cell donor.

15. (canceled)
